(19) United States
(12) Patent Application Publication
(10) Pub. No.: US 2012/0141543 A1
(43) Pub. Date: Jun. 7, 2012

(54) Carbon Nanotube Based Nanocomposites

(76) Inventors: Balaji Sitharaman, Lake Grove, NY (US); Antonios G. Mikos, Houston, TX (US); Lon J. Wilson, Houston, TX (US); Xinfeng Shi, Rochester, NY (US)

(21) Appl. No.: 11/972,886

(22) Filed: Jan. 11, 2008

Related U.S. Application Data
(60) Provisional application No. 60/884,582, filed on Jan. 11, 2007.

(51) Int. Cl. A61K 31/765; A61P 19/00; A61K 9/00; B82Y 5/00; B82Y 30/00

(52) U.S. Cl. 424/400; 424/78.31; 977/742; 977/750; 977/915

(57) ABSTRACT

Novel methods and compositions of nanocomposites are provided. One exemplary composition comprises a biocompatible polymer, such as polypropylene fumarate, and a carbon nanotube, such as a single walled carbon nanotube, an ultra-short carbon nanotube, or a substituted ultra-short carbon nanotube. An exemplary method comprises providing a biocompatible polymer and a carbon nanotube and combining a biocompatible polymer and a carbon nanotube to form a nanocomposite. Another exemplary method comprises providing a nanocomposite comprising a biocompatible polymer and a carbon nanotube and administering the composition to a subject.

### CROSS-REFERENCE TO RELATED APPLICATIONS

This application claims the benefit of U.S. Provisional Application Ser. No. 60/884,582 filed Jan. 11, 2007, which is incorporated by reference.

### STATEMENT OF GOVERNMENT INTEREST

This invention was made with government support under Grant Number R01 AR42639, awarded by the National Institutes of Health, Grant Number EEC-0118001 awarded by the National Science Foundation, and Grant Number 0624 awarded by the Robert A. Welch Foundation. The U.S. government has certain rights in the invention.

### BACKGROUND

In many cases, traditional treatments for bone defects involve the use of bone tissue from the same individual or from a bone bank, and permanent biomaterials, such as metals and ceramics. However, problems associated with, among other things, limited availability of autogenous tissue, potential disease transfer with allogenous tissue, and failure of permanent prostheses, have at least in part stimulated research towards the development of polymeric scaffolding materials that aid in bone tissue formation and regeneration.

Poly(propylene fumarate) (“PPF”) has been developed for use as, among other things, an injectable biocompatible polymer scaffold for bone tissue engineering applications. When so desired, PPF may be crosslinked with propylene fumarate-diacrylate (“PF-DA”), among other crosslinking molecules, to form a polymer network. However, even though the mechanical properties of crosslinked PPF may be comparable in many respects to those of trabecular bone, significant mechanical reinforcement is often needed for the use of the material as a scaffold having, among other properties, high porosity for guided tissue growth under load bearing conditions.

Single walled carbon nanotubes (SWNTs) have been considered as reinforcing fillers because of their superb mechanical properties (˜640 GPa in modulus and ˜40 GPa in tensile strength) and high aspect ratio. However, in many respects, a mechanical reinforcement may be questionable unless, among other things, an external loading force can be efficiently transferred to dispersed carbon nanotubes. Dispersion of SWNTs in a polymer remains a major challenge because, among other things, synthesized SWNTs usually exist as ropes of hundreds of individual nanotubes and also may aggregate into micron sized agglomerates due to, among other things, strong inter-tube van der Waals and π-π attraction (0.5 eV nm⁻¹). In many situations, such bundles or aggregates may cause slippage between nanotubes, become stress concentrators, or initiate cracks under applied loads. Furthermore, at the SWNT concentration at which enhanced mechanical properties may be achieved in a polymer, the SWNTs may show high viscosity, which may negatively affect the injectability of the polymer. The use of a surfactant or the covalent functionalization of SWNTs have been proven to be effective strategies in dispersing SWNTs in a polymer matrix.

### DRAWINGS

Some specific example embodiments of the disclosure may be understood by referring, in part, to the following description and the accompanying drawings.

FIG. 1 shows the dimensions of C₆₀, US tubes, and SWNTs.

FIGS. 2a-2f show the linear dynamic oscillatory shear viscoelastic response for uncrosslinked polymer and uncrosslinked nanocomposites with varying concentrations of C₆₀, SWNTs, and US tubes.

FIGS. 3a-3d show the mechanical properties of crosslinked nanocomposites at varying carbon nanostructure concentrations.

FIGS. 4a and 4b show the fracture surface of 0.5 wt % and 2 wt % US tube nanocomposites respectively, with US tubes broken and/or pulled out of the polymer matrix and covered by polymer.

FIGS. 5a and 5b show TEM images of 0.2 wt % SWNT and US tube nanocomposites, respectively.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure relates to compositions and methods related to carbon nanotubes. More particularly, the present disclosure relates to the preparation of a nanocomposite composition comprising ultra-short carbon nanotubes.

In certain embodiments, the present disclosure relates to nanocomposite compositions comprising a biocompatible polymer and a carbon nanotube. As used herein, the term “nanocomposite” is defined to include a composition wherein a nanoparticle, such as for example fullerenes (e.g., C₆₀) and carbon nanotubes, has been introduced into a macromolecule, such as for example a polymer. As used herein, the term “carbon nanotube” refers to a type of fullerene having an elongated, tube-like shape of fused five-member and six-member rings that is approximately 1 nm in diameter. Examples of carbon nanotubes that may be used in conjunction with the methods of the present disclosure may include, but are not limited to, ultra-short carbon nanotubes (US tubes) and substituted US tubes. As used herein, the term “US tubes” refers to ultra short carbon nanotubes with lengths from about 20 nm to about 100 nm. US tubes may be prepared by cutting SWNTs into ultra-short lengths. In certain embodiments, the carbon nanotubes used in the compositions of the present disclosure may comprise US tubes of length in the range of about 20 nm to about 80 nm. In certain embodiments, the carbon nanotubes used in the compositions of the present disclosure may comprise US tubes of a length of less than 100 nm. In certain embodiments, the carbon nanotubes used in the compositions of the present disclosure may comprise SWNTs of a length short enough such that the carbon nanotubes have adequate rheological properties to form injectable nanocomposites.

SWNTs, also known as single walled tubular fullerenes, are cylindrical molecules consisting essentially of sp² hybridized carbons. In defining the size and conformation of single-walled carbon nanotubes, the system of nomenclature described by Dresselhaus et al., Science of Fullerenes and Carbon Nanotubes, Ch. 19, ibid. will be used. Single walled tubular fullerenes are distinguished from each other by a double index (x,y), where x and y are integers that describe how to cut a single strip of hexagonal graphite such that its edges join seamlessly when the strip is wrapped onto the surface of a cylinder. When x=y, the resultant tube is said to be of the “arm-chair” or (x,x) type, since when the tube is cut perpendicularly to the tube axis, only the sides of the hexagons are exposed and their pattern around the periphery of the tube edge resembles the arm and seat of an arm chair repeated n times. When y=0, the resultant tube is said to be of the “zig-zag” or (x,0) type, since when the tube is cut perpendicular to the tube axis, the edge is a zig-zag pattern. Where x≠y and y≠0 the resulting tube has chirality. The electronic properties of the nanotube are dependent on, among other things, the conformation. For example, arm-chair tubes are metallic and have, among other things, extremely high electrical conductivity. Other tube types may be metallic, semi-metals or semi-conductors, depending on their conformation. Regardless of tube type, all SWNTs may have, among other things, extremely high thermal conductivity and tensile strength. In certain embodiments, the SWNT may be a cylinder with two open ends, a cylinder with one closed end, or a cylinder with two closed ends. In certain embodiments, an end of an SWNT may be closed by a hemifullerene, for example a (10,10) carbon nanotube can be closed by a 30-carbon hemifullerene. If the SWNT has one or two open ends, the open ends may have any valences unfilled by carbon-carbon bonds within the single wall carbon nanotube filled by bonds with hydrogen, hydroxyl groups, carboxyl groups, or other groups. SWNTs may also be cut into ultra-short pieces, thereby forming US tubes.

The carbon nanotubes useful in the compositions and methods of the present invention may be produced by any method known in the art. In certain embodiments, the carbon nanotubes, more particularly, the SWNTs, may be produced by the HiPco process or by electric arc discharge. A substantial amount previous research concerning the loading of SWNT samples has been performed with electric-arc discharge-produced SWNTs as opposed to other SWNT production methods, such as high-pressure carbon monoxide (HiPco). This is because, in many cases, arc-produced SWNTs have, among other things, a larger diameter than HiPco SWNTs (1.4 nm average diameter for arc vs. 1.0 nm diameter for HiPco) and arc SWNTs may contain more sidewall defects than HiPco SWNTs, thereby facilitating loading. For medical applications, however, the uniformity and purity of HiPco SWNTs may advantageous. Suitable commercially available carbon nanotubes may be obtained from Carbon Nanotechnologies Inc., Houston, Tex.

US tubes useful in the compositions and methods of the present invention may be formed by any method known in the art. In certain embodiments, such methods of producing US tubes may comprise cutting full-length SWNTs into short pieces by a four-step process. First, residual iron catalyst particles may be removed by oxidation via exposure to wet-air or SF₆ followed by a strong acid (HCl) treatment to extract the oxidized iron particles. The purified SWNTs may then be fluorinated by a gaseous mixture of 1% F₂ in He at elevated temperatures for up to 2 hours and cut into short pieces by pyrolysis under argon at 900° C. The fluorination reaction may produce F-SWNTs, with a stoichiometry of CFₓ (x<0.2), which may comprise bands of fluorinated-SWNT separated by regions of pristine SWNT. Pyrolysis under Ar, among other things, liberates volatile fluorocarbons, thereby cutting the SWNTs into pieces with lengths corresponding to the areas of pristine SWNT. While this method known in the art is effective at producing cut SWNTs, improvements can be made; for example, the separate purification step is unnecessary and can be eliminated. Such improvements, provided that they do not adversely affect the compositions and methods of the present invention, are considered within this spirit of the present invention.

In certain embodiments, a three-step process of producing US tubes may be used. First, as produced HiPco SWNTs may be fluorinated in a monel steel apparatus by a mixture of 1% F₂ in He at 100° C. for about 2 hours. During this process, both the SWNTs and the iron catalyst particles may become at least partially fluorinated. Subsequent exposure to concentrated HCl may substantially remove the fluorinated catalyst particles without affecting the F-SWNTs, which have a stoichiometry of ˜C₁₀F after the acid treatment. The now-purified F-SWNTs are cut into US tubes by pyrolysis under Ar at 900° C. In certain embodiments, the resulting US tubes have lengths ranging from 20-80 nm, with the majority being ˜40 nm in length. Utilizing this method, the amount of iron catalyst may be reduced from ˜25 mass percent in raw SWNTs to ˜1 mass percent for US tubes. Therefore, in certain embodiments, this method may be ideal for the purification of SWNTs, but only as a precursor to producing US tubes. This is because the fluorine remaining, after the HCl acid treatment, is difficult to remove, making the F-SWNTs only viable for subsequent cutting. Furthermore, the time to produce US tubes from SWNTs using this method may be significantly reduced.

The carbon nanotubes can be substituted or unsubstituted. By “substituted” it is meant that a group of one or more atoms is covalently linked to one or more atoms of the carbon nanotube. In certain embodiments, Bingel chemistry may be used to substitute the nanotube with appropriate groups. Examples of groups suitable for use in the compositions and methods of the present invention may include, but are not limited to, malonate groups, serinol malonates, groups derived from malonates, serinol groups, serinol amide, carboxylic acid, dicarboxylic acid, polyethyleneglycol (PEG), t-butylphenylene groups, and the like. The synthesis of substituted carbon nanotubes is described in further detail in X. Shi, J. L. Hudson, P. P. Spicer, J. M. Tour, R. Krishnamoorti, A. G. Mikos, Biomacromolecules 7, 2237-2242 (2006), the entire disclosure of which is incorporated herein by reference.

The nanocomposite compositions of the present disclosure also comprise a biocompatible polymer. Suitable biocompatible polymers may be, among other things, injectable and crosslinkable. An example of a suitable biocompatible polymer is poly(propylene fumarate) (“PPF”). Embodiments that comprise PPF may utilize a cross-linking agent comprising propylene fumarate-diacrylate (“PF-DA”).

In certain embodiments, the present disclosure provides a method comprising providing a biocompatible polymer, providing a carbon nanotube, and combining the biocompatible polymer with the carbon nanotube to form a nanocomposite.

In certain embodiments, the present disclosure provides a method comprising providing a nanocomposite comprising a biocompatible polymer and a carbon nanotube and administering the composition to a subject.

In certain embodiments, the compositions of the present invention may be delivered to a subject by injection. In certain embodiments, the nanocomposites of the present invention may be injected into living tissue and allowed to crosslink in vivo.

In certain embodiments, the nanocomposite compositions of the present disclosure may be used as an injectable scaffold for tissue engineering applications. Examples of such tissue engineering applications may include, but are not limited to, bone tissue engineering applications. In certain embodiments, the nanocomposite compositions of the present disclosure may be used in applications involving one or more of the following: implantology, bone surgery, traumatology, interventional radiology, and rheumatology.

To facilitate a better understanding of the present disclosure, the following examples of certain aspects of some embodiments are given. In no way should the following examples be read to limit, or define, the entire scope of the invention.

### EXAMPLES

The examples below utilized the following materials. Diethyl fumarate, hydroquinone, fumaric acid, acryloyl chloride, triethylamine, benzoyl peroxide (BP), and N,N-dimethyl-p-toluidine (DMT) were purchased from Sigma-Aldrich (St Louis, Mo.). Propylene glycol, zinc chloride, propylene oxide, pyridine, hydrochloric acid, sodium hydroxide, and sodium sulfate were purchased from Fisher-Acros (Fair Lawn, N.J.). C₆₀ (99.5+% purity) was purchased from Materials and Electrochemical Research Corporation (Tucson, Ariz.). Purified high-pressure CO converted (HiPco) SWNTs (iron content ˜2%) were obtained from Carbon Nanotechnologies (Houston, Tex.). All organic solvents were of reagent grade and were used as received.

### Example 1

### Synthesis of PPF and PF-DA

Poly(propylene fumarate) (PPF) and propylene fumarate-diacrylate (PF-DA) were synthesized according to methods described in A. K. Shung, M. D. Timmer, S. Jo, P. S. Engel, A. G. Mikos, J. Biomater. Sci. Polym. Ed. 95-108 (2002) and M. D. Timmer, C. G. Ambrose, A. G. Mikos, J. Biomed. Mater. Res. A 66, 811-818 (2003), the relevant portions of which are incorporated herein by reference. The polymer structures were confirmed by ₁H NMR, and the molecular weights were measured by gel permeation chromatography (GPC). A calibration curve generated from polystyrene standards (Fluka, Switzerland) with peak molecular weights ranging from 374 to 28,000 was used to determine PPF molecular weights. The PPF used for this study had a number average molecular weight (Mₙ) of 1600 Da and a weight average molecular weight (M_{w}) of 3500 Da. PF-DA had a molecular weight of 340.

### Example 2

### Synthesis of US Tubes and C₆₀

US tubes were synthesized by fluorination (100° C. for 2 hours and at a He:F₂ ratio of 99:1) followed by pyrolysis (1000° C. under Ar, 1 hour) of as-received SWNTs. This procedure resulted in cut SWNTs with lengths ranging mainly between 20 and 80 nm. As-received bulk solid C₆₀ was hand ground in an agate mortar prior to use for the different experiments.

### Example 3

### Surface Area Analysis

The surface areas of the carbon nanostructures were measured at 77 K with a Micromeritics ASAP 2010 Brunauer-Emmett-Teller (BET) surface analysis instrument (Micromeritics, Norcross, Ga.) using N₂ as adsorption gas. Measurements were repeated three times for each sample and the average of the three measurements was reported.

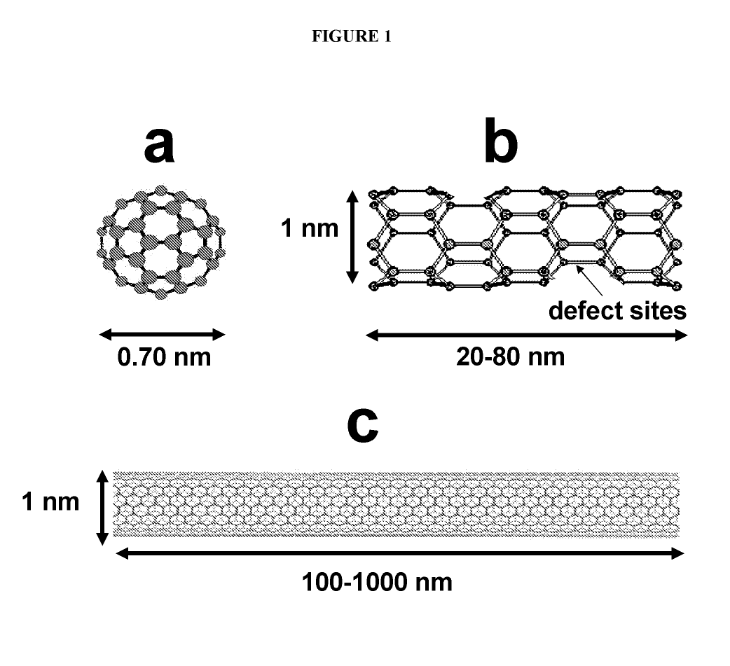

The measured BET surface area of the US tubes was 1023 m²/g, which is approximately double that of pristine SWNT (575 m²/g) and approximately four orders of magnitude that of C₆₀ (0.15 m²/g) (Table 1). This increased surface area for US tubes may arise from, among other things, the greater access to the interior hollow space of the SWNTs due to side-wall defects (and possibly end opening) during the fluorination/pyrolysis procedure. Such defects may allow small molecules such as N₂ used for BET surface area measurements to efficiently enter the interior space of the US tubes. It may seem counterintuitive to note that C₆₀, which has the smallest size (0.7 nm) among the carbon nanostructures, should have such a low surface area. This discrepancy is likely due to the existence of ground solid C₆₀ as a soft crystal nanoparticle of approximately 20 nm size. This crystal structure of these C₆₀ nanoparticles may prevent the access of the N₂ to all of the C₆₀ surfaces. FIG. 1 depicts the dimensions of C₆₀, US tubes, and SWNTs. The trends in size, surface area and aspect ratio characteristics of these three carbon nanostructures are summarized in Table 2. In general, the size (that is the diameter in case of C₆₀ and length in case of US tubes and SWNTs) and aspect ratio of the carbon nanostructures decrease in the order of SWNT>US tube>C₆₀. However, C₆₀ used in this study has the smallest surface area, while US tubes have the largest.

TABLE 1
BET Surface Areas for the Carbon Nanostructures (n = 3).

| | Measured |
|---|---|
| | Surface Area |
| Carbon Nanostructure | (m²/g) |
| C₆₀ | 0.15 ± 0.001 |
| SWNT | 574 ± 4 |
| US tube | 1023 ± 10 |

TABLE 2
Carbon Nanostructure Size, Surface Area and Aspect Ratio Trends.

| Characteristic | Trend |
|---|---|
| Size | SWNT > US tube > C₆₀ |
| Aspect Ratio | SWNT > US tube > C₆₀ |
| Surface Area | US tube > SWNT > C₆₀ |

### Example 4

### Nanocomposite Preparation

The nanocomposite samples were prepared by mixing PPF and PF-DA in chloroform at a mass ratio of 1:2.08. Carbon nanostructure samples were first dispersed in chloroform by high shear mixing for 5 minutes and sonicating for 15 minutes, then added into the PPF/PF-DA mixture at concentrations of 0-2.0 wt %. Prior to sample testing, chloroform was removed under reduced pressure, followed by drying. The preparation of nanocomposite samples is described in more detail in X. Shi, J. L. Hudson, P. P. Spicer, J. M. Tour, R. Krishnamoorti, A. G. Mikos, Nanotechnology 16, S531-S538 (2005), the entire disclosure of which is incorporated herein by reference.

### Example 5

### Rheological Testing

Rheological measurements were performed with an AR1000 rheometer (TA Instruments, New Castle, Del.) in an oscillatory shear mode at 25° C. Uncrosslinked polymer melt and nanocomposite melt samples were placed between a base plate and a cone geometry (60 mm diameter, 59 min cone angle, and 26 μm truncation). Each sample was examined as a function of the oscillatory strain frequency (ω) of 0.001-30 Hz using 0.01-0.1 strain amplitude and the complex viscosity magnitude (|η*|), storage modulus (G′), and loss modulus (G″) were recorded. The 0.01-0.1 strain amplitude was chosen to allow for rheological measurement in the linear dynamic range. For the nanocomposite melts, the strain amplitude used was at the low end of the reported range, while for the uncrosslinked polymer melt, the high end of the strain amplitude reported was employed. The viscous PPF polymer maintained the dispersion of carbon nanostructures within the polymer. Neither phase separation nor viscosity change (for a constant strain frequency) was observed during rheological analysis. For the SWNT nanocomposites, rheological measurements were performed only up to 0.2 wt % because solid like behavior commences at very low SWNT weight percentages (0.05-0.2 wt %).

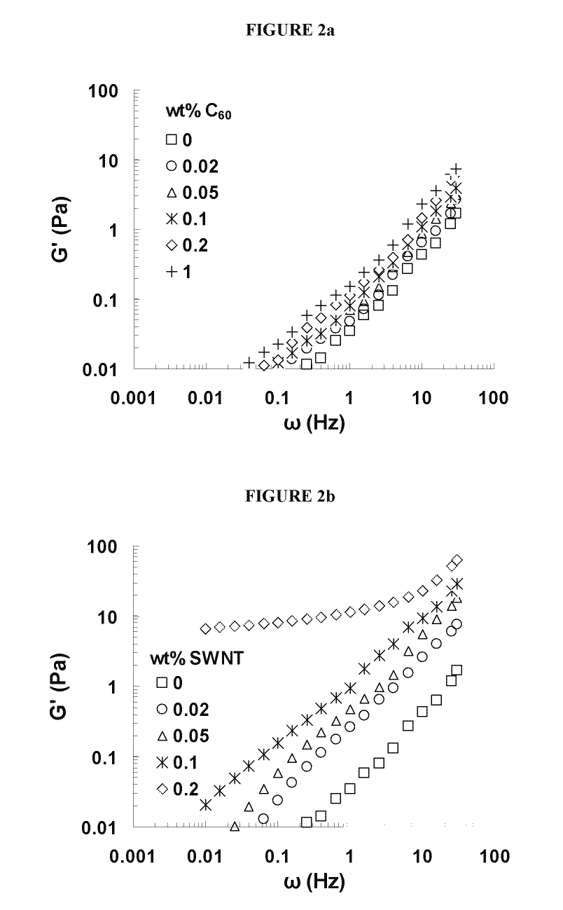

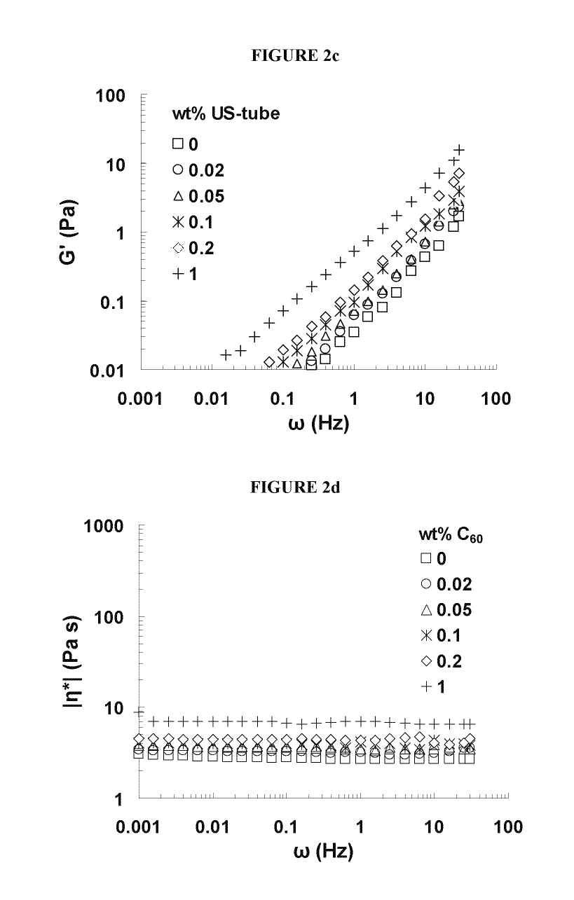

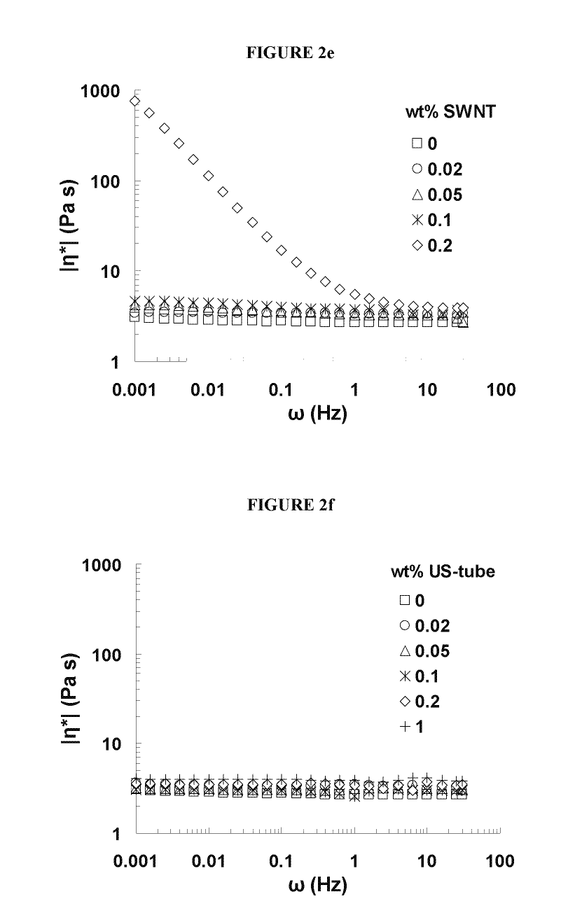

The linear dynamic oscillatory shear viscoelastic response for the uncrosslinked polymer and the uncrosslinked nanocomposites with varying concentrations of C₆₀, SWNTs, and US tubes are shown in FIGS. 2 (a)-(f), respectively. Table 3 reports the low frequency power law exponents for G′ and |η*| of these nanocomposites. While the elastic modulus of C₆₀ and US tube nanocomposites maintained viscous liquid-like behavior at all formulations (G′∝ω⁻²), the SWNT nanocomposites abruptly changed to solid-like behavior (G′∝ω^{˜0}) at 0.2 wt % SWNT loading. This implies that US tube and C₆₀ nanocomposites (up to 1 wt % loading) show lower viscosity than SWNT nanocomposites. The higher size and aspect ratio of the SWNTs lead to their entanglement when their concentrations are higher than the geometrical percolation threshold, thus contributing to increased viscosities. A decrease in size and aspect ratio may lead to, among other things, reduced entanglement and consequently lower viscosity. Both US tubes and C₆₀ have smaller sizes and aspect ratios than SWNTs. Thus, size and aspect ratio of the carbon nanostructures appear to be more important parameters than surface area for lower viscosity and hence good injectability.

TABLE 3
Low frequency power law exponents for uncrosslinked
nanocomposite formulations as a function of
Carbon Nanostructure concentration.

| | Conc. | Value of | Value of |
|---|---|---|---|
| Sample | (wt %) | α ^{a} | β ^{b} |
| PPF Polymer | 0 | 1.93 | 0.00 |
| C₆₀ | 0.02 | 1.92 | 0.00 |
| nanocomposites | 0.05 | 1.92 | 0.00 |
| | 0.1 | 1.93 | 0.00 |
| | 0.2 | 1.93 | 0.00 |
| | 1 | 1.93 | 0.00 |
| SWNT | 0.02 | 1.94 | 0.00 |
| nanocomposites | 0.05 | 1.93 | 0.00 |
| | 0.1 | 1.93 | 0.00 |
| | 0.2 | 0.05 | 0.87 |
| US tube | 0.02 | 1.93 | 0.00 |
| nanocomposites | 0.05 | 1.93 | 0.00 |
| | 0.1 | 1.93 | 0.00 |
| | 0.2 | 1.93 | 0.00 |
| | 1 | 1.93 | 0.00 |

^{a} G′ power law dependence (G′ ∝ ω^{α})
^{b} |η*| power law dependence (|η*| ∝ ω^{β})

### Example 6

### Thermal Crosslinking and Specimen Fabrication

The thermal crosslinking reaction of a nanocomposite formulation was triggered by the addition of 1 wt % BP (free radical initiator) and 0.15 wt % DMT (accelerator). BP was dissolved in diethyl fumarate at a concentration of 0.1 g/ml and added into the mixture, followed by the addition of DMT under vigorous stirring to initiate the polymerization. The polymeric mixture was filled into cylindrical glass vials 6.5 mm in diameter and 40 mm in length for compressive testing or injected into cylindrical glass tubes 3 mm in diameter and 150 mm in length for flexural testing. Both specimens were centrifuged at 721 g for 5 min to remove any air bubbles and then cured at 60° C. for 24 hours. A curing temperature of 60° C. was used in the study to ensure crosslinking of all test groups because, unlike SWNTs and US tubes, the C₆₀ nanocomposites did not substantially crosslink at 37° C. The specimens were recovered by breaking the glass container and then cutting the specimens to the proper lengths with a diamond saw (Model 650, South Bay Technology, San Clemente, Calif.). For compressive testing specimens, the length was twice their diameter (approximately 6.5 mm diameter and 13 mm length) and the two ends were flat and perpendicular to their long axis. The flexural testing specimens had dimensions of roughly 3 mm diameter and 60 mm length.

### Example 7

### Mechanical Testing

Compressive and flexural mechanical testing experiments were conducted at room temperature using an 858 Material Testing System mechanical testing machine (MTS Systems, Eden Prairie, Minn.). Five specimens were tested for each group (n=5). The results were expressed as means±standard deviation for n=5 for each sample group. Single-factor analysis of variance (ANOVA) was performed to assess the statistical significance within a data set. If the ANOVA test detected significance, Tukey's ‘Honestly Significantly Different’ (HSD) multiple-comparison test was used to determine the effects of the parameters examined. All comparisons were conducted at a 95% confidence interval (p<0.05).

American Society of Testing Materials (ASTM) Standard D695-02a was followed for the compressive testing. The prepared cylindrical specimens were compressed along their long axis until failure occurred, and the force and displacement were recorded throughout the compression. Stress and strain curves were generated based on the initial specimen dimensions. The slope of the initial linear portion of the curve gave the compressive modulus and a line drawn parallel to the slope defining the modulus, beginning at 1.0% strain (offset) gave the offset compressive yield strength (the stress at which the stress-strain curve intersected the line).

ASTM Standard D790-03 was followed for the flexural testing. The testing specimens were placed on a three-point bending apparatus with two supports spanning 40 mm from each other. A loading nose was loaded midway between the supports until the specimen failed. The recorded force and displacement were converted to a stress-strain curve and the flexural modulus was calculated from the stress-strain curve using methods similar to those used for compressive testing. The flexural strength was defined as the maximum stress carried by the specimen during the flexural testing.

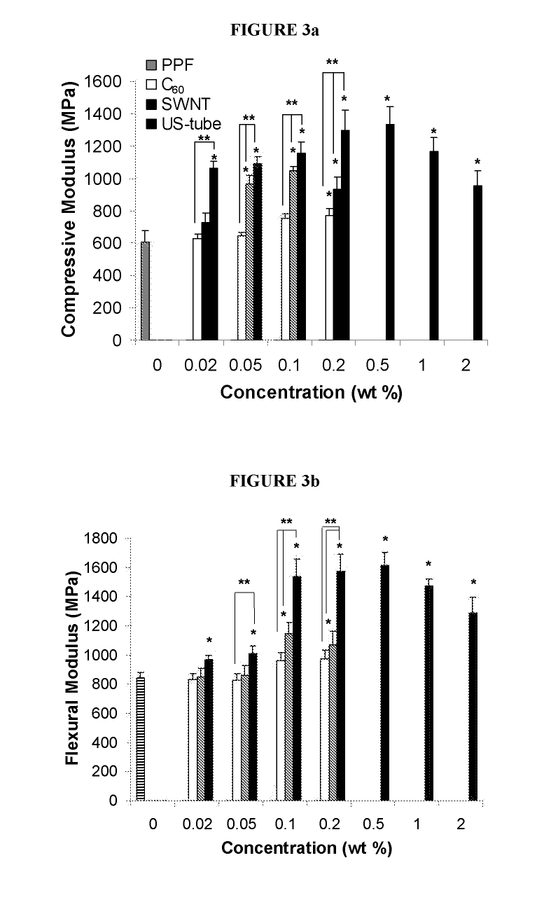

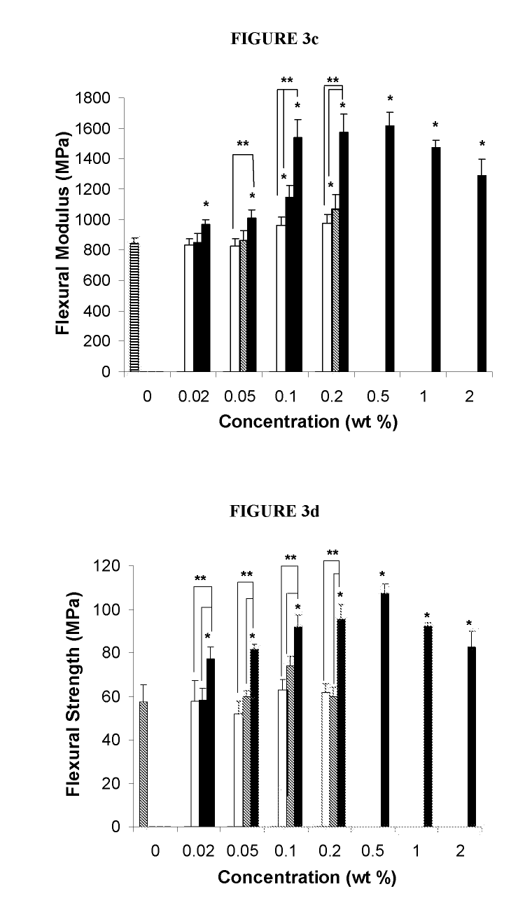

The mechanical properties of the crosslinked nanocomposites at varying carbon nanostructure concentrations are presented in FIGS. 3(a)-(d). C₆₀ nanocomposites show little difference in mechanical properties compared to plain PPF. For example, only the 0.2 wt % C₆₀ nanocomposite showed a significant increase of 26% in compressive modulus compared to plain PPF.

US tube nanocomposites showed the best mechanical enhancement effects. Unlike SWNT nanocomposites, the mechanical properties for US tube nanocomposites peaked at higher concentrations (0.5 wt %). Significant enhancements in compressive and flexural mechanical properties (up to 200%) were observed compared to the pure polymer. The mechanical properties of US tube nanocomposites were also higher than those for SWNT and C₆₀ nanocomposites.

Taken together, the mechanical properties for the three carbon nanostructure nanocomposites imply that US tubes and SWNTs contribute to better mechanical reinforcement than C₆₀. C₆₀ has a significantly lower surface area than both SWNTs and US tubes. Larger surface area may lead to better load transfer from polymer to nanostructures and hence better mechanical properties. Thus, surface area of carbon nanostructures may, among other things, be a more important parameter than size for mechanical reinforcement. Additionally, the fibril-like morphology of the SWNTs and US tubes may also contribute to the improved mechanical properties of the nanocomposites.

### Example 8

### Sol Fraction Study

The uncrosslinked polymer fraction of nanocomposites loaded with US tubes was assessed from sol fraction studies. Sol fraction analysis was performed on the US tube nanocomposites to verify whether the observed enhanced mechanical reinforcement resulted from higher cross-linking density and hence effective load transfer. The chemical cutting procedure employed to synthesize the US tubes gives rise to a number of defect sites. These defect sites can be easily be oxidized by atmospheric O₂ and introduce carboxy or hydroxy functional groups. These functional groups may, then in turn interact chemically with the polymer and increase the crosslinking density of the polymer.

An approximately 0.5 g sample was weighed (accuracy of Wᵢ=0.001 g) into a vial with 20 ml of methylene chloride. Next the vial was sealed and placed on a shaker table (80 rpm) at room temperature for 7 days. The solid sample was then filtered with a weighed filter paper (Wₚ). The retained material on the filter paper was dried at 60° C. for 1 h and kept at room temperature for another 1 h and then weighed again (Wₚ₊ₛ). The sol fraction was calculated using the following equation for each group (n=5):

Sol fraction=(Wᵢ−(Wₚ₊ₛ−Wₚ))/Wᵢ×100%.

The results were expressed as means±standard deviation for n=5 for each sample group. Single-factor analysis of variance (ANOVA) was performed to assess the statistical significance within a data set. If the ANOVA test detected significance, Tukey's ‘Honestly Significantly Different’ (HSD) multiple-comparison test was used to determine the effects of the parameters examined. All comparisons were conducted at a 95% confidence interval (p<0.05).

The sol fractions measured for the different US tube nanocomposites varied from 0.13±0.02 to 0.18±0.07 and no significant difference was found amongst these different samples (data not shown). These results are similar to the sol fraction values obtained with pristine SWNT nanocomposites, Shi et al., Nanotechnology 16, S531-S538 (2005), but different from functionalized SWNT nanocomposites which showed increase in crosslinking density with SWNT concentration, Shi et al., Biomacromolecules 7, 2237-2242 (2006). These results indicate that US tubes do not significantly change the PPF crosslinking density. Thus, the observed mechanical reinforcement cannot be attributed to changes in crosslinking density and maybe due to other attributes, such as increased US tube-polymer interfacial area.

### Example 9

### Scanning Electron Microscopy (SEM)

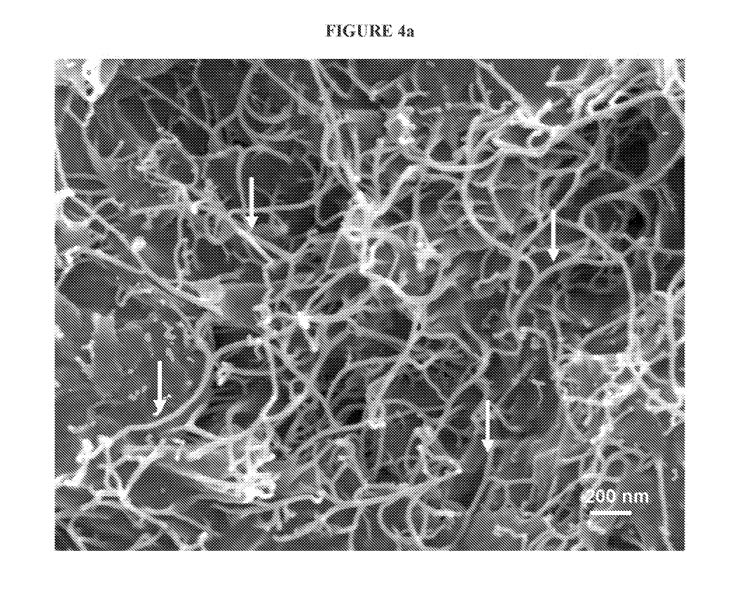

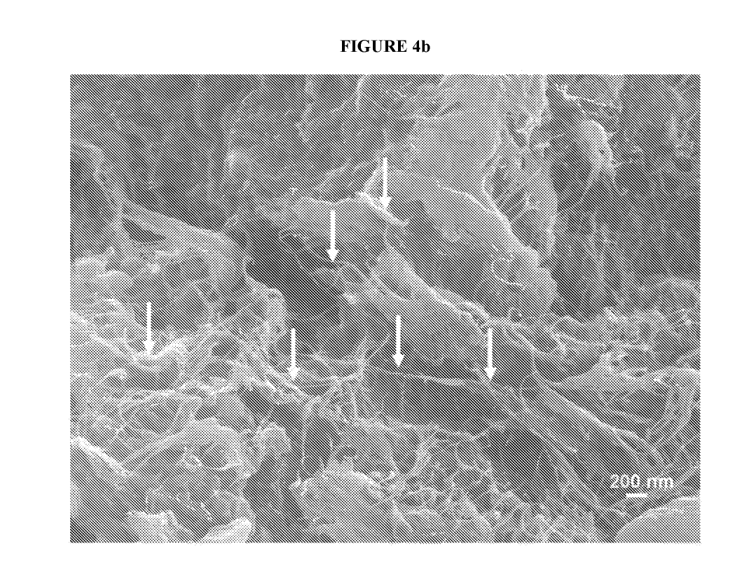

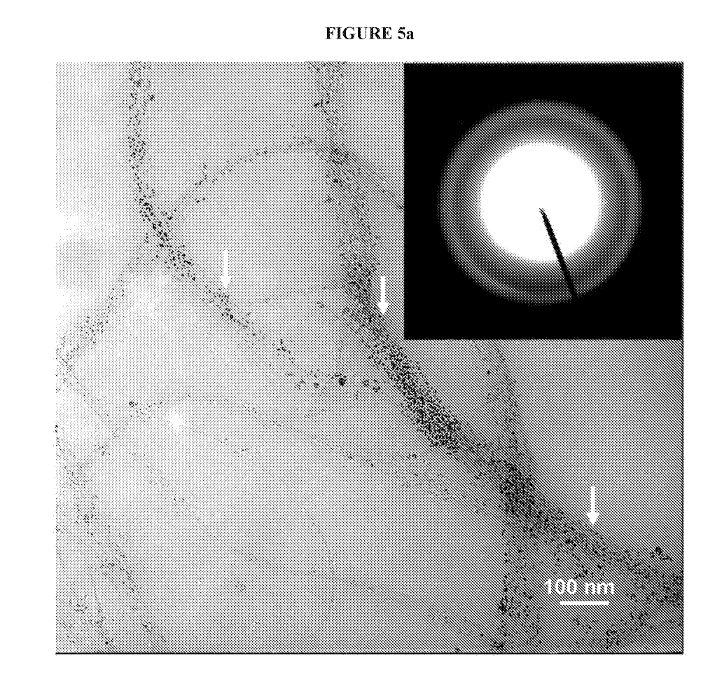

SEM imaging was carried out on a JEOL 6500F scanning electron microscope (SEM; JEOL USA, Peabody, Mass.) at an accelerating voltage of 15 kV. Fracture surfaces of tested specimens from mechanical testing were sputter coated with gold prior to SEM imaging. FIGS. 4a and 4b show the fracture surface of 0.5 wt % and 2 wt % US tube nanocomposite with US tubes broken and/or pulled out of the polymer matrix and covered by polymer. SEM images further confirmed that US tubes were, among other things, well-dispersed in the crosslinked polymer matrix.

### Example 10

### Transmission Electron Microscopy (TEM)

Crosslinked nanocomposite samples were sectioned to thin specimens (50-100 nm in thickness) using a Leica Ultracut microtome (Leica, Vienna, Austria). Specimens were then mounted on a copper grid coated with amorphous carbon-holey film. A few drops of methylene chloride were added on the nanocomposite loaded holey carbon film sample and dried to view the carbon nanostructure-polymer interface. TEM imaging was carried out on a JEOL 2000 FX electron microscope operating at 200 kV. TEM analysis included conventional, high-resolution TEM imaging, and selected area electron diffraction (SAED).

TEM analysis was also used to reveal the nature of the interface for US tube and SWNT nanocomposites as the surface area of the carbon nanostructure appeared to play a fundamental role in the reinforcement mechanism. TEM analysis included conventional and high-resolution TEM imaging and selected-area electron diffraction (SAED). TEM studies were performed with caution to minimize the effect of heating and irradiation influence by the electron beam on the specimens. No solvent dissipation process was observed.

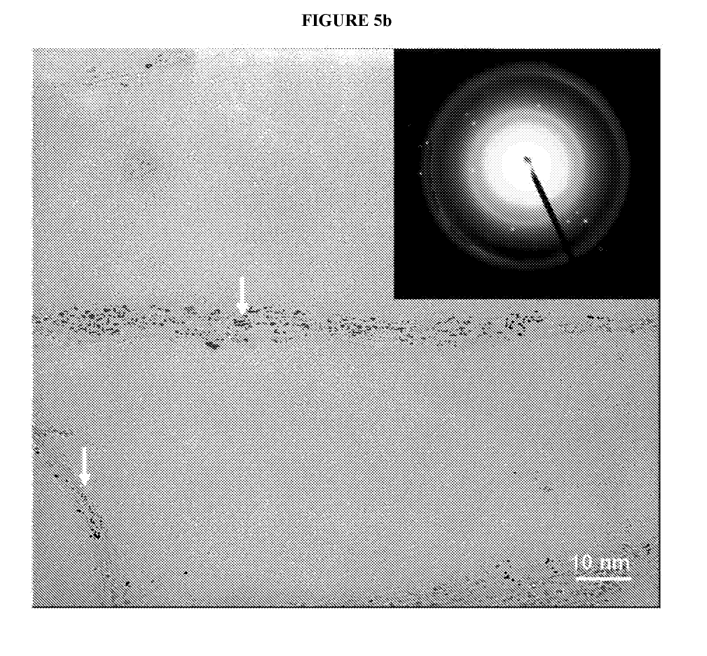

FIGS. 5a and 5b present the TEM images of 0.2 wt % SWNT and US tube nanocomposites respectively. The images show a thin coating of the crosslinked polymer over the carbon nanostructure surfaces. The SAED patterns (shown in insets), taken at the same area as the images, display spot diffraction patterns indicating crystallinity at the nanotube-polymer interface. The images suggest that PPF crystallizes on the surface of both pristine SWNTs, as well as US tubes, implying that PPF can crystallize on both smooth and defect-induced carbon nanotube surfaces.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A nanocomposite composition comprising: a biocompatible polymer; and at least one carbon nanotube.

2. The composition of claim 1 wherein the at least one carbon nanotube comprises at least one carbon nanotube selected from the group consisting of: a single walled carbon nanotube, an ultra-short carbon nanotube, and a substituted ultra-short carbon nanotube.

3. The compositions of claim 1 where in the at least one carbon nanotube comprises at least one carbon nanotube selected from the group consisting of: an ultra-short carbon nanotube, and a substituted ultra-short carbon nanotube.

4. The composition of claim 1 wherein the biocompatible polymer is crosslinkable.

5. The composition of claim 1 further comprising a crosslinker.

6. The composition of claim 1 wherein the biocompatible polymer comprises poly(propylene fumarate).

7. The composition of claim 1 further comprising propylene fumarate-diacrylate, and wherein the biocompatible polymer comprises poly(propylene fumarate).

8. The composition of claim 1 wherein the composition is injectable.

9. A method comprising:
providing a biocompatible polymer and at least one carbon nanotube; and
combining a biocompatible polymer and the at least one carbon nanotube to form a nanocomposite.

10. The method of claim 9 wherein the carbon nanotube comprises at least one carbon nanotube selected from the group consisting of a single walled carbon nanotube, an ultra-short carbon nanotube, and a substituted ultra-short carbon nanotube.

11. The method of claim 9 wherein the biocompatible polymer comprises poly(propylene fumarate).

12. The method of claim 9 further comprising: injecting the nanocomposite into living tissue; and allowing the nanocomposite to crosslink in vivo.

13. The method of claim 9 wherein the nanocomposite is injectable.

14. A method comprising:
providing a nanocomposite comprising a biocompatible polymer and at least one carbon nanotube; and
administering the nanocomposite to a subject.

15. The method of claim 14 wherein the at least one carbon nanotube comprises at least one carbon nanotube selected from the group consisting of a single walled carbon nanotube, an ultra-short carbon nanotube, and a substituted ultra-short carbon nanotube.

16. The method of claim 14 wherein the biocompatible polymer comprises poly(propylene fumarate).

17. The method of claim 14 wherein the step of administering the nanocomposite to the subject comprises injecting the nanocomposite into the subject.

18. The method of claim 14 wherein the step of administering the nanocomposite to the subject is performed to treat a bone defect.
